(19) United States
(12) Patent Application Publication
(10) Pub. No.: US 2014/0302101 A1
(43) Pub. Date: Oct. 9, 2014

(54) NATURAL HAIRSTYLE HOLDING PRODUCT CONTAINING LEVAN AS A FILM-FORMER

(71) Applicant: John Carson, Union City, NJ (US)

(72) Inventor: John Carson, Union City, NJ (US)

(21) Appl. No.: 13/857,233

(22) Filed: Apr. 5, 2013

(51) Int. Cl. A61K 8/73; A61Q 5/06

(52) U.S. Cl. CPC A61K 8/73; A61Q 5/06
USPC 424/401; 424/70.13; 424/47

(57) ABSTRACT

A hairstyle holding composition formed of natural or naturally derived ingredients, the composition comprising a hairstyle holding resin, wherein the hairstyle holding resin consists essentially of a low viscosity levan, wherein the levan produces a low intrinsic viscosity dispersion in water having particle sizes in the range of from about 50 to about 200 nm, and wherein the composition further includes at least one natural or naturally derived additive, wherein the at least one additive is selected from the group consisting of natural oils, natural antimicrobial substances, a natural fragrance, a natural emulsifier, a natural viscosity controller and a natural liquefied or gaseous form hydrocarbon to serve as a propellant.

### TECHNICAL FIELD

The disclosure herein is directed to a hairstyle holding product, formulated, for instance, as a hair spray or a hair mousse, that utilizes levan as a film-forming agent and in which non-natural, i.e., synthetic components are avoided in favor of naturally occurring and/or naturally derived materials. That is, notwithstanding the occasional unavoidable presence of a minimal amount of synthetic materials which do not, in any case, have any material effect on the characteristics of the claimed composition, including the method of its making, the hair-style holding product as described and claimed herein is deemed to be produced substantially with the use of natural and/or naturally derived components.

### BACKGROUND OF THE INVENTION

Hair sprays are fine sprays combining a film-forming material which, when applied to a subject's hair, forms a film on the hair, and a carrier liquid which is usually volatile and that evaporates, thus leaving the film-forming material on the hair. A hair mousse, on the other hand, is a foam form of the combination of a film-forming material and a foam forming agent, which also contains volatile components which evaporate leaving a film on the subject's hair.

The film-forming material used in prior art hair sprays and hair mousses is typically a synthetic resin. The resin may be dissolved in a carrier comprised of alcohol, water and alcohol mixtures and possibly other volatile mixtures that evaporate, leaving the film resin on the hair. Sprays and mousses may contain various other additives, such as silicones, bactericides, emulsifiers and other non-natural, manufactured, synthetic chemicals. They may, furthermore, include herbal products in addition to the above-described synthetic products. Such prior art sprays and mousses may contain, furthermore, synthetic fragrance generating materials. The aerosol version of such sprays and mousses may additionally include synthetic material propellants mixed with the film-forming material, such as hydrofluorocarbons and dimethyl ether, as well as natural hydrocarbons, which are typically used in aerosol hair sprays.

Many of the synthetic materials used in prior art hair sprays or hair mousses are non-biodegradable materials that may contaminate a water supply and may have harmful effects to anyone or any animal that ingests them. They may also act as severe eye irritants.

The film-forming material used in the presently disclosed formulation is not a synthetic material, but rather it is levan, which is described further below. There are examples in the prior art of uses of levan in hair care products or hair sprays. Non-naturally occurring resins are used in such levan hair care products, as disclosed in US 2001/0022967, 2006/0029561 and 2008/0102051, whereas the present formulation, as indicated above, does not include such resins. Other uses of levan in hair care products are described in U.S. Pat. Nos. 7,572,933; 7,179,451; and 7,438,898, as well as in Published U.S. Patent Application No. 2011/0311463 A1 published Dec. 22, 2011.

### SUMMARY OF THE INVENTION

The hairstyle holding formulation described herein utilizes levan as the film-forming component. The composition may be formulated as, for example, a hair spray or mousse. The formulation does not include any synthetic resin to create a hair-holding film. In fact, in the formulation of the claimed composition, as far as possible, the use of any synthetic, i.e., non-natural, component is avoided. That is, only natural or naturally derived materials are included in the formulation, with the exception of possibly some very minor amounts of synthetic materials whose presence cannot otherwise be avoided. Notwithstanding the above, however, the presence of any such non-natural, i.e., synthetic material(s) does not have any material effect on either the characteristics or performance of the formulation as herein described and claimed. As used herein, the terms “natural” and “naturally derived” should be taken to have their ordinary and accepted meanings to those having at least an ordinary level of skill in this field of art, i.e., the terms are meant to exclude the presence of any significant amount of a synthetic, i.e. non-natural, material in whatever component is being discussed.

As discussed further below in additional detail, the film-forming material is a low viscosity levan that is typically combined with one or more additional components added to provide certain desirable characteristics to the hairstyle holding product according to the present invention. These additional components may include, for example, any of a natural oil to add sheen to the hair, natural substances for antimicrobial purposes, a natural fragrance, in some cases a natural emulsifier and a natural viscosity controller. Furthermore, when the composition is formulated in the form of, for example, an aerosol hair spray, a propellant may be utilized that is a natural liquefied or gaseous form hydrocarbon.

In one embodiment the invention is directed to a hairstyle holding composition formed of natural or naturally derived ingredients, wherein the composition comprises a hairstyle holding resin. The hairstyle holding resin consists essentially of a low viscosity levan wherein the low viscosity levan produces a low intrinsic viscosity dispersion in water wherein the dispersion has particle sizes in the range of from about 50 to about 200 nm.

In another embodiment, the low viscosity levan in present in the composition in an amount of between about 2 and about 20 weight %.

In another embodiment, of the hairstyle holding composition, the composition further includes at least one additional natural or naturally derived additive, the additive being selected from the group consisting of natural oils, natural antimicrobial substances, natural fragrance, a natural emulsifier, a natural viscosity controller, and a natural liquefied or gaseous form hydrocarbon propellant.

In another embodiment, the composition includes at least one natural oil in an amount of between about 0.1 and about 20% by weight of the composition.

In another embodiment the at least one natural oil is selected from the group consisting of argan oil, almond oil, avocado oil, apricot kernel oil, babassu oil, broccoli seed oil, castor oil, corn oil, cotton seed oil, canola oil, coconut oil, daikon radish seed oil, grape seed oil, flax seed oil, hemp seed oil, jojoba oil, kukui nut oil, macadamia nut oil, oat oil, olive oil, peanut oil, palm kernel oil, rice bran oil, rapeseed oil, safflower seed oil, sesame seed oil, shea butter, soybean oil, sunflower seed oil, turnip seed oil, wheat germ oil, cranberry oil, green tea oil, pomegranate oil, raspberry oil and tea tree oil.

In another embodiment the composition includes at least one natural antimicrobial substance in an amount of between about 0.1 and about 5 weight % of the composition.

In another embodiment the at least one natural antimicrobial substance is selected from the group consisting of benzoic acid, hydroxybenzoic acid, Origanum Vulgare (Oregano) Leaf Extract, Thymus Vulgaris (Thyme) Extract, Cinnamomum Zeylanicum (Cinnamon) Bark Extract, Olea Europaea (Olive) Leaf Extract, Rosmarinus Officinalis (Rosemary) Leaf Extract, Mentha Piperita (Peppermint) Leaf Extract, Lavandula Angustifolia (Lavender) Flower Extract, Hydrastis Canadensis (Goldenseal) Root Extract, Citrus Medica Limonum (Lemon) Peel extract, Caprylyl Glycol, Phenoxyethanol.

In another embodiment the composition includes at least one natural solvent selected from the group consisting of Ethanol, Glycerin and/or 1,3 Propane Diol which can function as a solvent and an antimicrobial agent in an amount of between about 2.0 and about 25 weight % of the composition.

In another embodiment the composition includes at least one natural fragrance in an amount of between about 0.1 and about 5 weight % of the composition.

In another embodiment the composition includes at least one natural emulsifier in an amount of between about 0.1 and about 10 weight % of the composition.

In another embodiment the natural emulsifier is selected from the group consisting of lecithin, glyceryl oleate, glyceryl stearate, glyceryl palmitate, glyceryl linoleate, polyglyceryl oleate, polyglyceryl stearate, polyglyceryl palmitate, polyglyceryl linoleate, phospholipids, Glycerin (and) Glycine max (Soybean) Seed Extract, Sunflower Seed Extract, alkylpolyglucosides, phytosterols, fatty acids, fatty acid soaps and sucrose esters.

In another embodiment the composition includes at least one natural viscosity controller in an amount of from about 0.1 to about 10 weight % of the composition.

In another embodiment the at least one natural viscosity controller is a gum, a clay or a combination thereof.

In another embodiment the at least one natural viscosity controller is a gum selected from the group consisting of guar gum, agar agar, carrageenan, acacia, cellulosics, xanthan, starch, gum arabic, gellan, locust bean, alginates, tragacanth, inulin and pullulan.

In another embodiment the at least one natural viscosity controller is a clay selected from the group consisting of kaolin, bentonite, montmorillonite and hectorite.

In another embodiment the composition is mixed with at least one of a naturally occuring liquified hydrocarbon propellant and a gaseous hydrocarbon propellant to permit dispending the composition in spray form.

In another embodiment the propellant is present in the composition in an amount of between about 2 and about 30 weight % of the composition.

In another embodiment the composition is produced in a form selected from the group consisting of a pump hair spray, an aerosol hair spray, a mousse, a gel, a liquid, a cream, a lotion, a serum and an emulsion.

In another embodiment the invention is directed to a hairstyle holding composition formed of natural or naturally derived ingredients. The composition comprises a hairstyle holding resin consisting essentially of from about 2 to about 20 weight % of a low viscosity levan. The low visciosity levan produces a low intrinsic dispersion in water wherein the dispersion has particle sizes in the range of from about 50 to about 200 nm, and wherein the composition further includes one additional natural or naturally derived additive, which at least one additive is selected from the group consisting of natural oils, natural antimicrobial substances, natural fragrance, a natural emulsifier, a natural viscosity controller, and a natural liquefied or gaseous form hydrocarbon propellant.

In still another embodiment, the composition described in the paragraph above is produced in a form selected from the group consisting of a pump hair spray, an aerosol hair spray, a mousse, a gel, a liquid, a cream, a lotion, a serum and an emulsion.

### DESCRIPTION OF PREFERRED EMBODIMENTS

The low viscosity levan used in the present hair-style holding product as a film former, e.g., in a hair spray, hair mousse or other hair control product, is a polymer of fructose, which is fruit sugar, and it is generically called a fructan. Fructans are built up of fructose residues, normally with a sucrose unit at what would otherwise be the reducing terminus. The linkage position of the fructose residues determines the type of the fructan. Linkage normally occurs at one of the two primary hydroxyls (OH-1 or OH-6). In the case of levan, the fructosyl residues are linked by Beta-2,6 linkages. Levan is thus Beta 2,6 fructan. In sum, then, levan is a homopolysaccharide composed of D-fructofuranosyl residues joined by 2,6 linkages.

Levan can be produced by a variety of bacteria, such as Microbacterium laevaniformans, Streptococcus salivarius, Pseudomonas syringae, Bacillus subtilus and Zymomonas, or created by breaking down other kinds of plant fructans such as soybean mucilage. The levan for use in the presently described hair-style holding formulation is a low viscosity levan that can be produced by Bacillus subtilus and Zymomonas, wherein it forms 50-200 nm diameter spheres in a water solution or dispersion having pendant fructan polymer segments that aid in the dispersion of the particles in water and promote a stable water dispersion. The levan is typically referred to as “low viscosity” levan due to the fact that it has an unusually low intrinsic viscosity (typically less than 0.2 dl/gm) in water as compared to about 1 dl/gm for polysaccharides such as amylose and about 20 dl/gm for extended chain polymers such as alginates or galactomannans. In addition, it has been shown that a thin coating of the low viscosity levan as described herein dries to a hard, shiny finish that is smooth to the touch and not tacky. At the present time only the low viscosity levan produced by B. subtilis is readily commercially available, however the Zymomonas bacterium is also believed capable of producing such low viscosity levan. The low viscosity levan from B. subtilis is presently commercially available from Montana Polysaccharides, Inc. located in Winnsboro, S.C.

The B. subtilis-produced low viscosity levan from Montana Polysaccharides, Inc. is made by fermentation of sucrose, whereupon the levan produces a dispersion in the water. The levan can be further refined into a solid material by filtering to remove the bacteria and then drying the dispersion or by precipitating the polymer with water soluble organic solvents such as ethanol, isopropanol, acetone, methyl ethyl ketone, etc. at a ratio of about 2:1 (solvent: levan dispersion), cooling the mixture and then filtering out and drying the precipitate. Thus, it was very surprising that physically stable dispersions could be made from a water dispersion of levan and ethanol at ratios up to and over 1:1. The addition of ethanol to the levan water dispersion is very desirable because it acts as a preservative and improves the ability of the formula to incorporate additional components such as fragrance components, shine enhancing oils, surfactants and antimicrobial agents. The levan, as a water, or water/solvent dispersion, functions as a film former that, when applied to and dried on hair, holds the hair in place (i.e. in a particular style). Any other additives in the hairstyle holding formulation as presently described and claimed herein are natural or naturally derived, and thus are not synthetic substances.

Levan in water solution/dispersion is subject to bacterial degradation and, thus, should be protected by antimicrobial agents. In an embodiment of the present formulation, that may be accomplished using ethanol, glycols, natural herbal bactericides or combinations of these materials. Natural or naturally derived antimicrobials are also useful for inclusion in the presently described formulation. Several examples of natural bactericides that may be used include, but are not limited to, benzoic acid, hydroxybenzoic acid, Origanum Vulgare (Oregano) Leaf Extract, Thymus Vulgaris (Thyme) Extract, Cinnamomum Zeylanicum (Cinnamon) Bark Extract, Olea Europaea (Olive) Leaf Extract, Rosmarinus Officinalis (Rosemary) Leaf Extract, Mentha Piperita (Peppermint) Leaf Extract, Lavandula Angustifolia (Lavender) Flower Extract, Hydrastis Canadensis (Goldenseal) Root Extract, Citrus Medica Limonum (Lemon) Peel extract, Caprylyl Glycol, Phenoxyethanol, Ethanol, Glycerin, 1,3 propane diol, and the like.

In certain embodiments of the present formulation, in order to provide additional sheen to the hair, natural oils may be added. A natural fragrance may also be added, whereas synthetic fragrances should be entirely avoided. Some hair sprays or mousses or other hair care formulations may require an emulsifier to provide adequate combination and stability of these lipophilic ingredients, in which instance, a natural emulsifier may be used. In addition, it may be desirable to control the viscosity and/or the rheology of a mousse or personal care product such as a serum, cream or gel. This may be accomplished by using a gum, or clay, or combinations of the two ingredients in a manner that is well understood in the art of hair care products.

In other embodiments of the present hairstyle holding product, in order to produce an aerosol spray version of a hair spray, the propellant that is used would be a natural liquefied or gaseous form hydrocarbon. This could be used alone or combined with naturally occurring compressed gases, such as carbon dioxide, nitrogen, argon, etc.

It is of course understood that in a hair spray or mousse of all naturally occurring ingredients, as indicated above trace amounts of some non-natural ingredients may be present. Such synthetic components, however o not have any material effect on any of the characteristics of the formulation.

Depending on the desired level of hold for the hair spray, mousse or other hair holding product, the amount of levan by weight in the mixture with other ingredients varies in the range of about 2% to about 20%. The percentage by weight of other major ingredients in the mixture depends on regulations of a local jurisdiction, such as state or country, regarding the total amount of a volatile organic substances (voc) permitted in a sprayable product. These amounts are discussed further below.

If herbal or botanical ingredients are used, depending upon the type of such ingredients, their percentage weight in the present formulation may range from about 0.1% to about 1%. If lipophilic materials or fragrance oils are used, their weight range in the formulation may range from about 0.1% to about 5%. If natural oils are used, their weight in the formulation may range from about 0.1% to about 20%. When natural antimicrobial substances are included in the formulation, their weight may range from about 0.1% to about 5%. When natural emulsifiers are included in the formulation, their weight may range from about 0.1% to about 10%. When a viscosity controller is used in the formulation, its weight may range from about 0.1% to about 10%. Furthermore, when a propellant is included, such propellant varies by weight in the range of from about 2% to about 30%.

Natural oils useful for inclusion in the presently described hair style holding formulation may include, but are not limited to such as : argan oil, almond oil, avocado oil, apricot kernel oil, babassu oil, broccoli seed oil, castor oil, corn oil, cotton seed oil, canola oil, coconut oil, daikon radish seed oil, grape seed oil, flax seed oil, hemp seed oil, jojoba oil, kukui nut oil, macadamia nut oil, oat oil, olive oil, peanut oil, palm kernel oil, rice bran oil, rapeseed oil, safflower seed oil, sesame seed oil, shea butter, soybean oil, sunflower seed oil, turnip seed oil, wheat germ oil, cranberry oil, green tea oil, pomegranate oil, raspberry oil, tea tree oil, and the like.

Natural emulsifiers useful for inclusion in the presently described formulation may include, but are not limited to such as lecithin, glyceryl oleate, glyceryl stearate, glyceryl palmitate, glyceryl linoleate, polyglyceryl oleate, polyglyceryl stearate, polyglyceryl palmitate, polyglyceryl linoleate, phospholipids, Glycerin (and) Glycine max (Soybean) Seed Extract, Sunflower Seed Extract, alkylpolyglucosides, phytosterols, fatty acids, fatty acid soaps, sucrose esters, and the like.

Viscosity control agents useful in the presently described formulation may include, but are not limited to clays such as kaolin, bentonite, montmorillonite and hectorite, and gums such as guar gum, agar agar, carrageenan, acacia, cellulosics, xanthan, starch, gum arabic, gellan, locust bean, alginates, tragacanth, inulin, pullulan, and the like.

The mixture, appropriately adjusted in a well-known manner to be dispensed, can be used in a pump spray or aerosol spray version as, for example, a hair spray or a hair mousse. The hair mousse comes out as foam, while the spray comes out as a liquid spray. It has been surprisingly discovered that low viscosity levan readily allows the production of low viscosity water solutions or dispersions that can be sprayed. In contrast, biopolymers (such as Xanthan, Pullulan and levan from other bacterial sources) produce water solutions that are high in viscosity and indeed these biopolymers are used in the Personal Care and Food industries as viscosity builders.

The following formulas are presented as non-limiting examples of preferred embodiments of the instant formulation and are intended to be illustrative of, but not limiting to, the concepts described herein. As used in the examples, the term “Levan” is specifically meant to refer to the low-viscosity levan as described above.

### EXAMPLES 1 and 2

| INGREDIENTS | w/w % Example 1 | w/w % Example 2 |
|---|---|---|
| Deionized water | 75.8 | 67.8 |
| Ethanol | 20.0 | 20.0 |
| Levan (dried polymer) | 4.0 | 12.0 |
| Grapeseed Oil | 0.1 | — |
| Fragrance oil blend | 0.1 | 0.2 |
| | 100.0 | 100.0 |

Example 1 is soft to regular hold non-aerosol or pump hair spray, while Example 2 is a hard hold non-aerosol pump spray formula. Interestingly and surprisingly both of these formulas provide good shine on hair. Both formulas dry quickly to non-tacky films within 1 to 3 minutes. The ethanol is used in these examples as a natural antimicrobial agent and a rapid evaporation solvent. Both examples can be re-styled once dried using a comb or brush, although this does cause some reduction in holding because welds and contact joins are broken. Restyling can also be accomplished by wetting the comb or hair while restyling. This procedure rehydrates the levan, allowing restyling and causes very little loss in style holding once the hair dries again.

### EXAMPLES 3 and 4

| INGREDIENTS | w/w % Example 3 | w/w % Example 4 |
|---|---|---|
| Deionized water | 75.5 | 67.3 |
| Ethanol | 20.0 | 20.0 |
| Levan (dried polymer) | 4.0 | 12.0 |
| Grapeseed Oil | — | 0.2 |
| Suprapein * | 0.5 | 0.5 |
| | 100.0 | 100.0 |

*INCI NAME: Origanum Vulgare (Oregano) Leaf Extract CAS No: 84012-24-8; Thymus Vulgaris (Thyme) Extract CAS No: 84929-51-1; Cinnamomum Zeylancum (Cinnamon) Bark Extract CAS No: 84649-98-9; Olea Europaea (Olive) Leaf Extract CAS No: 8060-29-5; Rosmarinus Officinalis (Rosemary) Leaf Extract CAS No: 84604-14-8; Mentha Piperita (Peppermint) Leaf Extract CAS No: 84082-70-2; Lavandula Angustifolia (Lavender) Flower Extract CAS No: 90063-37-9; Hydrastis Canadensis (Goldenseal) Root Extract CAS No: 84603-60-1; Citrus Medica Limonum (Lemon) Peel extract CAS No: 8008-56-8

Examples 3 and 4 are non-aerosol hair sprays showing the use of a natural preservative blend (i.e., Suprapein) in addition to ethanol that is added in these formulas to promote more rapid drying. The odor was initially very strong and “herbal” due to the Suprapein, however the intensity of the odor diminished over time to an acceptable level.

### EXAMPLES 5 and 6

| INGREDIENTS | w/w % Example 5 | w/w % Example 6 |
|---|---|---|
| Deionized water | 71.8 | 68.8 |
| Ethanol | 20.0 | 20.0 |
| Levan (dried polymer) | 4.0 | 8.0 |
| Grapeseed Oil | 0.1 | — |
| Fragrance oil blend | 0.1 | 0.2 |
| Propellant A-46 | 4.0 | 3.0 |
| | 100.0 | 100.0 |

Examples 5 and 6 are aerosol formulas. They were packaged into glass pressure bottles with suitable valves and actuators in order to observe spray characteristics, stability and ingredient compatibility. Both examples sprayed acceptably and produced a hard, non-tacky film after drying when sprayed on glass.

### EXAMPLES 7 and 8

| INGREDIENTS | w/w % Example 7 | w/w % Example 8 |
|---|---|---|
| Deionized water | 89.4 | 76.5 |
| Ethanol | — | 10.0 |
| Levan (dried polymer) | 4.0 | 6.0 |
| Lecithin | 1.0 | — |
| Lauryl Glucoside | — | 2.0 |
| Grapeseed Oil | 0.1 | — |
| Suprapein | 0.5 | 0.5 |
| Propellant A-46 | 5.0 | 5.0 |
| | 100.0 | 100.0 |

Example 7 produced a wet, heavy foam that had a somewhat sticky feeling. It gives a light style hold, while Example 8 made a better, more “puffy” foam and produces a stiffer style hold.

### EXAMPLES 9 and 10

| INGREDIENTS | w/w % Example 9 | w/w % Example 10 |
|---|---|---|
| Deionized water | 63.9 | 57.1 |
| Ethanol | 20.0 | 20.0 |
| Levan (dried polymer) | 15.0 | 10.0 |
| Lauryl Glucoside | — | 2.0 |
| Xanthan Gum | 0.8 | 0.8 |
| Olive Oil | — | 10.0 |
| Fragrance oil blend | 0.3 | 0.1 |
| | 100.0 | 100.0 |

Example 9 is a hair serum providing stiff hold and shine to hair. Example 10 is a hair holding cream/gel that provides fairly stiff hold and leaves the hair very shiny and with a smooth oily feel.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A hairstyle holding composition formed of natural or naturally derived ingredients, said composition comprising a hairstyle holding resin, wherein the hairstyle holding resin consists essentially of a low viscosity levan and wherein the low viscosity levan produces a low intrinsic viscosity dispersion in water wherein said dispersion has particle sizes in the range of from about 50 to about 200 nm.

2. The hairstyle holding composition according to claim 1, wherein the levan is present in the composition in an amount of between about 2 and about 20 weight %.

3. The hairstyle holding composition according to claim 1, wherein the composition further includes at least one additional natural or naturally derived additive, said at least one additive being selected from the group consisting of natural oils, natural antimicrobial substances, a natural solvent, a natural fragrance, a natural emulsifier, a natural viscosity controller and a natural liquefied or gaseous form hydrocarbon propellant.

4. The hairstyle holding composition according to claim 3, wherein the composition includes at least one natural oil in an amount of between about 0.1 and about 20 weight % of said composition.

5. The hairstyle holding composition according to claim 4, wherein the at least one natural oil is selected from the group consisting of argan oil, almond oil, avocado oil, apricot kernel oil, babassu oil, broccoli seed oil, castor oil, corn oil, cotton seed oil, canola oil, coconut oil, daikon radish seed oil, grape seed oil, flax seed oil, hemp seed oil, jojoba oil, kukui nut oil, macadamia nut oil, oat oil, olive oil, peanut oil, palm kernel oil, rice bran oil, rapeseed oil, safflower seed oil, sesame seed oil, shea butter, soybean oil, sunflower seed oil, turnip seed oil, wheat germ oil, cranberry oil, green tea oil, pomegranate oil, raspberry oil and tea tree oil.

6. The hairstyle holding composition according to claim 3, wherein the composition includes at least one natural antimicrobial substance in an amount of between about 0.1 and about 5 weight % of said composition.

7. The hairstyle holding composition according to claim 6, wherein the at least one natural antimicrobial substance is selected from the group consisting of benzoic acid, hydroxybenzoic acid, Origanum Vulgare (Oregano) Leaf Extract, Thymus Vulgaris (Thyme) Extract, Cinnamomum Zeylanicum (Cinnamon) Bark Extract, Olea Europaea (Olive) Leaf Extract, Rosmarinus Officinalis (Rosemary) Leaf Extract, Mentha Piperita (Peppermint) Leaf Extract, Lavandula Angustifolia (Lavender) Flower Extract, Hydrastis Canadensis (Goldenseal) Root Extract, Citrus Medica Limonum (Lemon) Peel extract, Caprylyl Glycol, Phenoxyethanol.

8. The hairstyle holding composition according to claim 3, wherein the composition includes at least one natural solvent selected from the group consisting of Ethanol, Glycerin and 1,3 Propane Diol in an amount of between 2.0 and about 25 weight % of said composition.

9. The hairstyle holding composition according to claim 3, wherein the composition includes at least one natural fragrance in an amount of between about 0.1 and about 5 weight % of said composition.

10. The hairstyle holding composition according to claim 3, wherein the composition includes at least one natural emulsifier in an amount of between about 0.1 and about 10 weight % of said composition.

11. The hairstyle holding composition according to claim 9, wherein the natural emulsifier is selected from the group consisting of lecithin, glyceryl oleate, glyceryl stearate, glyceryl palmitate, glyceryl linoleate, polyglyceryl oleate, polyglyceryl stearate, polyglyceryl palmitate, polyglyceryl linoleate, phospholipids, Glycerin (and) Glycine max (Soybean) Seed Extract, Sunflower Seed Extract, alkylpolyglucosides, phytosterols, fatty acids, fatty acid soaps and sucrose esters.

12. The hairstyle holding composition according to claim 3, wherein the composition includes at least one natural viscosity controller in an amount of from about 0.1 to about 10 weight % of said composition.

13. The hairstyle holding composition according to claim 11, wherein the at least one natural viscosity controller is a gum, a clay or a combination thereof.

14. The hairstyle holding composition according to claim 12, wherein the at least one natural viscosity controller is a gum selected from the group consisting of guar gum, agar agar, carrageenan, acacia, cellulosics, xanthan, starch, gum arabic, gellan, locust bean, alginates, tragacanth, inulin and pullulan.

15. The hairstyle holding composition according to claim 12 wherein the at least one natural viscosity controller is a clay selected from the group consisting of kaolin, bentonite, montmorillonite and hectorite.

16. The hairstyle holding composition according to claim 3, wherein the composition is mixed with at least one of a naturally occurring liquefied hydrocarbon propellant and a gaseous hydrocarbon propellant to permit dispensing said composition in spray form.

17. The hairstyle holding composition according to claim 15, wherein said propellant is present in an amount of between about 2 and about 30 weight % of said composition.

18. The hairstyle holding composition according to claim 1 wherein the composition is produced in a form selected from the group consisting of a pump hair spray, an aerosol hair spray, a mousse, a gel, a liquid, a cream, a lotion, a serum and an emulsion.

19. A hairstyle holding composition formed of natural or naturally derived ingredients, said composition comprising a hairstyle holding resin, wherein the hairstyle holding resin consists essentially of from about 2 to about 20 weight % of a low viscosity levan, wherein the low viscosity levan produces a low intrinsic dispersion in water wherein said dispersion has particle sizes in the range of from about 50 to about 200 nm and wherein the composition further includes at least one additional natural or naturally derived additive, said at least one additive being selected from the group consisting of natural oils, natural antimicrobial substances, a natural fragrance, a natural emulsifier, a natural viscosity controller and a natural liquefied or gaseous form hydrocarbon to serve as a propellant.

20. The hairstyle holding composition according to claim 18 wherein the composition is produced in a form selected from the group consisting of a pump hair spray, an aerosol hair spray, a mousse, a gel, a liquid, a cream, a lotion, a serum and an emulsion.
